(19) United States
(12) Patent Application Publication
(10) Pub. No.: US 2002/0188265 A1
(43) Pub. Date: Dec. 12, 2002

(54) Leak eze cap

(76) Inventor: Michael Douglas Law, Brentwood, TN (US)

Correspondence Address: Michael D. Law, 1747 Charity Drive, Brentwood, TN, 37027, US

(21) Appl. No.: 10/040,712

(22) Filed: Jan. 7, 2002

Related U.S. Application Data
(60) Provisional application No. 60/260,769, filed on Jan. 10, 2001.

(51) Int. Cl. A61F 13/15

(52) U.S. Cl. 604/368; 604/385.24

(57) ABSTRACT

This product is a disposable male incontinence device that is lightweight, easy-to-use and discreet for the customer. A low-tension elastic band holds the cap snugly around the head of the penis. The inner lining is constructed of highly absorbent cotton fiber with a one-way flow for moisture, similar to that found in feminine napkins and baby diapers. Because the inner lining is highly absorbent, the moisture is maintained in the lining, and away form the skin, which allows for repeated use after urination, if the customer chooses.

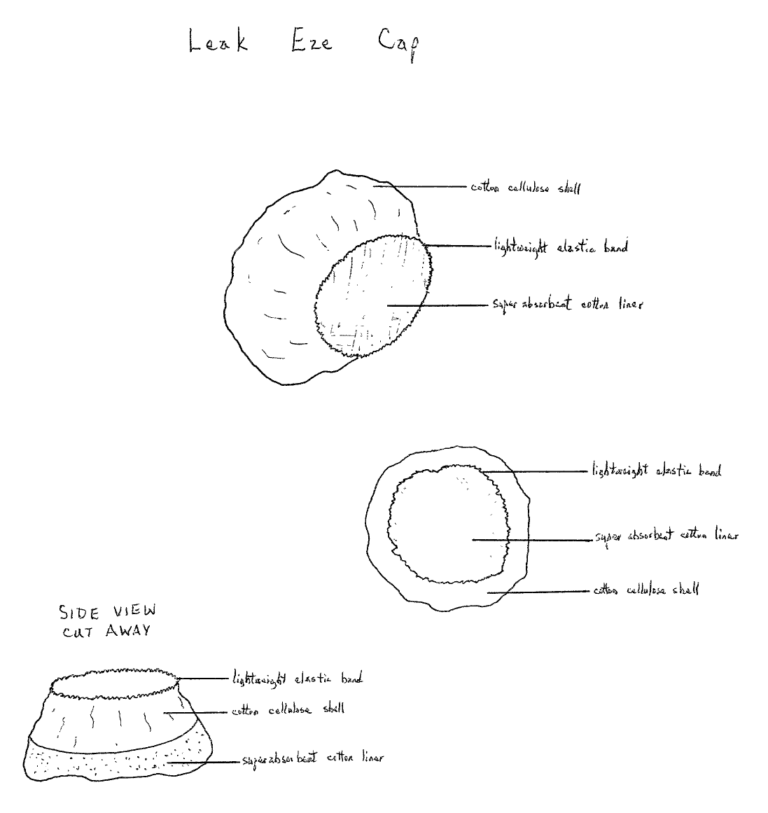

I HAVE DESIGNED A PRODUCT THAT HAS A PROVISIONAL PATENT NUMBER. THE APPLICATION No. IS: 60/260,769. THE FILING DATE IS: Jan. 10, 2001, AND THE TITLE OF THE PRODUCT IS: LEAK EZE CAP.

### DESCRIPTION OF THE INVENTION

THIS PRODUCT WILL BE USED BY MALES OF ALL AGES, BUT PREDOMINATELY BY THE MALE POPULATION OVER THE AGE OF FORTY (40) YEARS. THIS PRODUCT WAS CREATED TO ASSIST MEN WHO EXPERIENCE THE DISCOMFORT AND EMBARRASSMENT OF POST VOID AND POSTM COITUS LEAKAGE.

NUMEROUS STUDIES HAVE BEEN PUBLISHED SUPPORTING THE FACT THAT AS MEN AGE, THEY EXPERIENCE FREQUENT OCCASSIONS WHERE THEY LEAK URINE AFTER VOIDING AND SEMINAL FLUID AFTER INTERCOURSE. UNLIKE THE ADULT DIAPERS THAT ARE DESIGNED FOR INCONTINENCE, THIS PRODUCT IS DESIGNED FOR “LEAKAGE”. IN ADDITION TO ADULT DIAPERS, THERE ARE BEHAVIOR MODIFICATION PROGRAMS AND PRESCRIPTION DRUGS TO TREAT INCONTINENCE, BUT NEITHER ARE APPROPRATE FOR THE ANNOYING LEAKAGE THAT CAUSES EMBARRASSING STAINS IN A MAN'S UNDERGARMENTS AND TROUSERS. THERE ARE OTHER PATENTED PRODUCTS DESIGNED FOR “LEAKAGE”, BUT THEY ARE NOT AS EASY TO USE AS MY PRODUCT.

THE EASIEST WAY TO DESCRIBE MY PRODUCT IS TO COMPARE IT TO A SURGICAL BONNET. IMAGINE THIS SURGICAL BONNET IS LINED WITH A FEMALE ULTRA THIN SANITARY NAPKIN. THE LIGHTWEIGHT CELLULOSE FIBER OF THE SHELL MAKES THE PRODUCT EASY TO USE, AND THE SUPER ABSORBENCY OF THE ULTRA THIN NAPKIN PROTECTS THE MAN FROM IRRITATION BROUGHT ON BY DAMPNESS NEXT TO THE SKIN, BECAUSE ALL MOISTURE IS ABSORBED LIKE A FEMININE NAPKIN, AND A DRY LAYER REMAINS NEXT TO THE SKIN. THE PRODUCT IS HELD IN PLACE WITH A LOOSE ELASTIC BAND THAT IS NOT CONSTRICTING ON THE PENIS.

UTILIZING U. S. CENSUS BUREAU DATA (YEAR 2000), I NOTED THAT THERE ARE OVER FIFTY MILLION (50M) MEN IN THE UNITED STATES BETWEEN THE AGES OF FORTY (40) AND EIGHTY-FIVE (85). MANY OF THESE MEN, ESPECIALLY IN THE YOUNGER AGE GROUPS, DO NOT NEED ADULT DIAPERS NOR PRESCRIPTION DRUGS (WITH THEIR POSSIBLE SIDE EFFECTS) TO CONTROL THIS ANNOYING AND EMBARRASSING PROBLEM. MY PRODUCT OFFERS THEM AN INEXPENSIVE, EASY-TO-USE SOLUTION TO THIS PROBLEM.

1. I CLAIM THE CELLULOSE OUTER SHELL, THE SUPER ABSORBENT COTTEN LINER AND THE LIGHTWEIGHT ELASTIC BAND. THESE COMPONENTS ALLOW FOR THE PRODUCT TO BE USED DISCREETLY AND EASILY IN PUBLIC RESTROOMS AND EFFECTIVELY PREVENTS EMBARRASSING AND ANNOYING LEAKS THAT SOIL UNDERWEAR AND OUTER GARMENTS.
